(19) United States
(12) Patent Application Publication
(10) Pub. No.: US 2006/0178596 A1
(43) Pub. Date: Aug. 10, 2006

(54) Cutaneous indentation sensory testing device

(75) Inventors: Daniel R. Robichaud II, Worcester, MA (US); Marco Cannella, Philadelphia, PA (US); Peter Grigg, Worcester, MA (US); Geoffrey M. Bove, Kennebunkport, ME (US)

Correspondence Address: Roger P. Zimmerman;Mirick, O'Connell, DeMallie & Lougee, LLP, 100 Front Street, Worcester, MA, 01608-1477, US

(73) Assignee: University of Massachusetts, Boston, MA (US)

(21) Appl. No.: 11/339,027

(22) Filed: Jan. 25, 2006

Related U.S. Application Data
(60) Provisional application No. 60/646,770, filed on Jan. 25, 2005.

(51) Int. Cl. A61B 19/00; A61B 5/103

(52) U.S. Cl. 600/553; 600/587

(57) ABSTRACT

In one embodiment, the present invention provides a sensory testing system. In another embodiment, the present invention provides a method of using a sensory testing system to determine sensory pressure thresholds. In a further embodiment, the present invention provides a method of diagnosing a condition characterized by impaired neural function by using a sensory testing system to determine sensory pressure thresholds.

### RELATED APPLICATIONS

This application claims benefit of U.S. Provisional Patent Application 60/646,770 filed Jan. 25, 2005, the entire contents of which are incorporated by reference for all purposes.

### GOVERNMENT SUPPORT

This invention was supported, in whole or in part, by grants NS-10783 and AR-48925 from the National Institutes of Health. The United States government has certain rights in the invention.

### BACKGROUND OF THE INVENTION

Sensory testing of the skin is done to investigate possible compromised touch or pain sensation. Such testing can be used to detect peripheral neuropathies of various origins, such as diabetes mellitus.

Sensory testing of the skin is commonly performed by applying filament stimulators, such as von Frey hairs or Symmes-Weinstein filaments, to the skin of the patient or test subject. The filament is advanced past the point of contact to compress the skin until the filament buckles. The patient or subject reports whether or not the resulting compression is detected and whether the sensation is painful. When the filament buckles, the compression force that the filament exerts on the skin is approximately independent of the degree of buckling, and is dependent on the material composition and the structure of the filament, i.e., its diameter, length and composition. Thus, the physician sequentially applies filaments of increasing stiffness and consequently exerting greater compressive force, during the course of testing the sensibility at a given point on the skin's surface. The force required to produce a criterion response, such as a report of pain, is recorded and the process repeated at another point within the test area on the skin.

### SUMMARY OF THE INVENTION

In one embodiment, the present invention provides a sensory testing system. In another embodiment, the present invention provides a method of using a sensory testing system to determine sensory pressure thresholds. In a further embodiment, the present invention provides a method of diagnosing a condition characterized by impaired neural function by using a sensory testing system to determine sensory pressure thresholds. The method of the present invention facilitates rapid and accurate sensory testing by eliminating the time consuming use of manually operated von Frey hairs.

In preferred embodiments, the invention provides a sensory testing system including a test filament having a proximal end and a distal end, the proximal end for engaging a test subject, the test filament further associated with an axis substantially parallel to the probe, a motor having a shaft moveable along the axis, the shaft further coupled to the distal end of the filament, the motor further capable of moving the shaft at a constant force without requiring a force measuring device in communication with the test filament; and a controller for controlling the operation of the motor. Preferably the test filament remains substantially rigid over a determined range of applied forces and the motor is a linear motor.

Generally, the system further includes a displacement sensor for measuring a displacement of the shaft. In preferred embodiments, the controller is a digital computer processing machine-readable instructions. The system can be used for testing human subjects, such patients suspected of suffering from conditions such as peripheral neuropathy or diabetes. The system also can be used for testing nonhuman subjects such as veterinary patients or laboratory test animals.

In preferred embodiments, the present invention provides a method for performing sensory measurements on a subject, the method comprising the steps of receiving an initialization instruction from a controller; receiving an advancement instruction for causing a shaft to increment (“ramp up”) the operating force until a detection signal is received; receiving the detection signal; stopping advancement of the shaft in response to the detection signal; and recording the force applied at the time of the detection signal.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic illustration of an embodiment 100 of the method of the present invention, showing the steps of selecting a test area on the subject 102, entering selected parameters into a sensory testing system 104, applying a probe of the sensory testing system to the test area 106, receiving a stop signal 108 and recording the pressure exerted by the probe on the test area.

FIG. 2A is a diagrammatic illustration of an embodiment of the sensory test system of the present invention suitable for use with non-human subject.

FIG. 2B is a diagrammatic illustration of the relationship of linear motor 206, filament retainer 204, displacement sensor 210, movable base 212 and cable 216 in one embodiment of the sensory test system of the present invention.

FIG. 3A and FIG. 3B are graphic representations of results obtained using an embodiment of the sensory test system of the present invention, showing in FIG. 3A a plot of load versus time, and in FIG. 3B a plot of displacement versus time.

FIG. 4 is a diagrammatic illustration of another embodiment of the sensory test system of the present invention suitable for use with human subject.

FIG. 5 is a diagrammatic representation of an embodiment of a suitable computer 400 for use in the sensory testing system of the present invention.

FIG. 6A is a schematic illustration of a high level flow diagram showing four phases associated with an exemplary embodiment of the method of the present invention for using an embodiment of a sensory testing system 239 to perform sensory testing on a subject.

FIG. 6B and FIG. 6C are schematic illustrations that together illustrate the steps of FIG. 6A in detail.

FIG. 7 is a schematic illustration of a networked embodiment of a sensory testing system 239.

### DETAILED DESCRIPTION OF THE INVENTION

In preferred embodiments, the present invention provides a sensory testing system for determining thresholds for tactile, or haptic, sensation in human or animal subjects. In preferred embodiments, the sensory testing system includes a linear DC motor having a shaft and a single test filament mounted on the end of the shaft that is positioned toward the subject during testing. The linear DC motor is operatively linked to a length encoder and to a digital motor controller, which are both operatively linked to a computer that executes a program that controls the movement of the motor shaft and detects its position. During testing the system advances the test filament under program control. The force applied by the motor is increased according to a defined function, preferably a ramp function. The applied force and the displacement of the motor shaft are monitored and stored under program control.

Human subjects communicate the chosen sensory endpoint, e.g., detectible touch or pain, by generating a detection signal that stops the motor and causes the current producing the compression force of the motor to be recorded. Alternatively, especially in studies of nonhuman subjects, the chosen sensory endpoint is communicated by withdrawal of the body part, such as a foot, that is being tested. Withdrawal of the body part can be detected by the sensory testing system using a displacement transducer. The force that was applied just prior to the communication that the chosen sensory endpoint has been reached is taken as the threshold force. Values of threshold forces are stored in data files, and can be used to create a map of threshold forces superimposed on an image (schematic diagram or video image) of the particular body part being tested.

The linear motor is operated in a mode that simulates force control. In this mode, the computer moves the motor shaft and attached test filament to maintain a selected force that is determined by the control program. The force exerted by the test filament is independent of how the probe is held by the operator because the motor is operated in a force control mode.

As used herein, “computer” refers to a digital computer capable of executing programs, and having a processor, memory and input and output devices. Preferably the computer is capable of sensing and manipulating its surroundings by detecting signals and generating signals using the input and output devices. Suitable computers are portable general purpose computers such as laptops and tablet computers, as well as personal digital assistants such as Pocket PCs, Palm and the like. “Subject” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

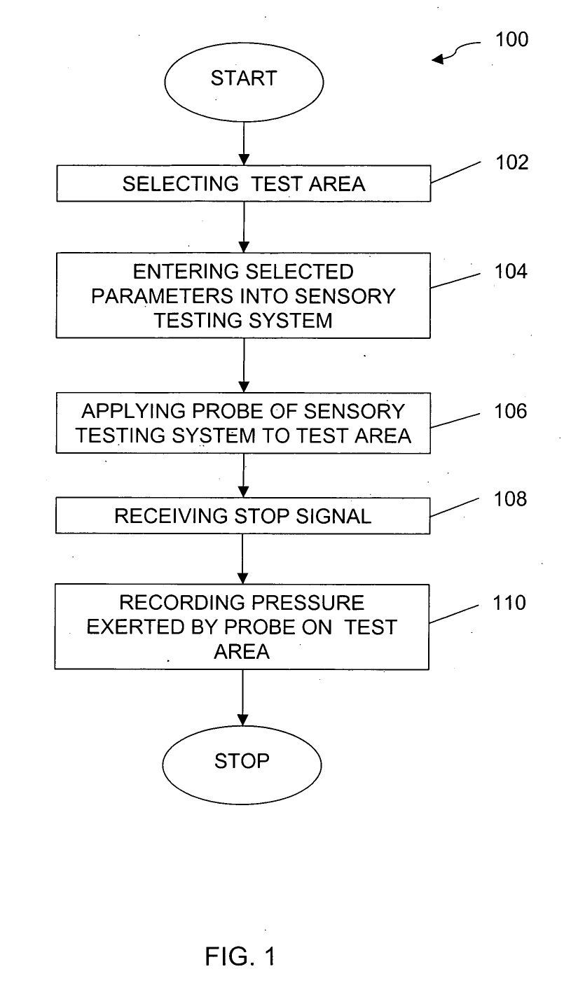

FIG. 1 illustrates an exemplary method for performing sensory measurements using embodiments of the invention. A test area is identified on the surface of a subject (per step 102). For example, the skin on the lateral surface of a subject's arm may be identified as the test area for performing sensory measurements. Next, test parameters are identified and entered into the sensory testing system using an input device (per step 104). In one preferred embodiment, the test parameters are entered using a keyboard as an input device. In other embodiments, test parameters are entered using a keypad in response to prompts or chosen from choices provided on a responsive display, such as a touch sensitive screen.

In preferred embodiments, the measurement of sensory function is performed using a linear motor having an extendable shaft with a probe attached thereto at the end of the shaft nearer the subject (see step 106). The probe is attached to the end of the shaft that extends toward the subject at an increasing force, suitably increasing as a ramp function. When a subject senses pressure, preferably a criterion level of pressure, such as painful pressure, the subject reports the sensation using a device that produces a signal that can be detected by the sensory testing system. In a preferred embodiment, the signal is an electrical signal produced by closing a switch, e.g. a human subject depressing a hand-held stop button. In other embodiments, a non-human subject can be trained to report a criterion level of pressure using an appropriate device.

The signal is received by the sensory test system and directly or indirectly acts to stop the travel of the shaft and probe combination toward the subject (per step 108). When the stop signal is received by the system, the pressure exerted by the probe'is recorded by the sensory test system (per step 110). In certain embodiments, the sensory test system further measures and records the distance traveled by the shaft.

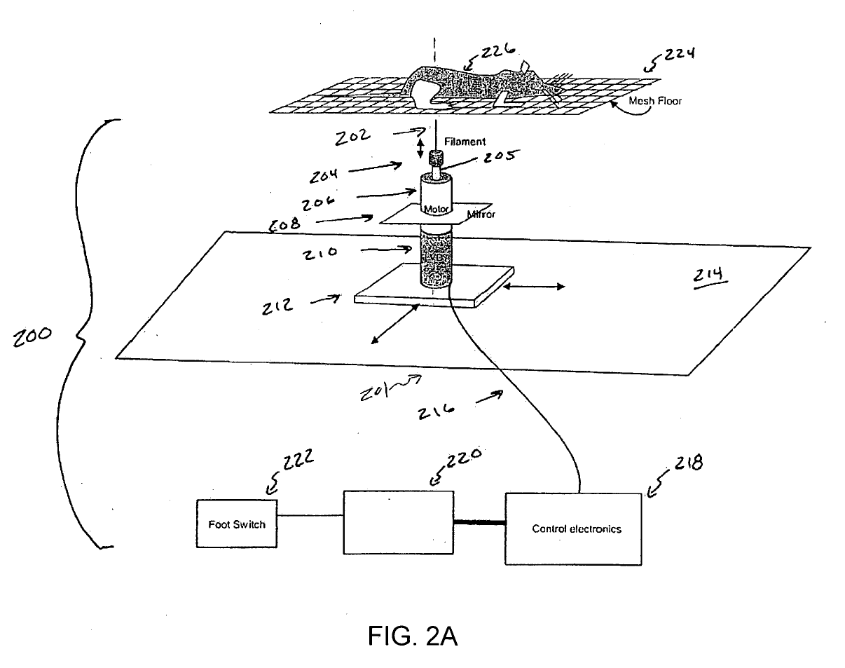

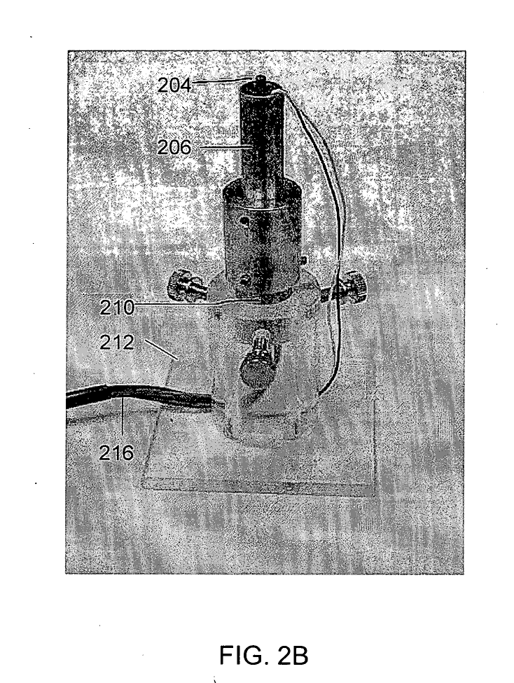

FIG. 2A contains a schematic representation of a preferred embodiment of a system 200 for making measurements of sensory function of a subject in a laboratory environment, such as a non-human subject, e.g., a rat. System 200 includes a test filament 202, a retainer 204, a motor shaft 205, a motor 206, a mirror 208, a displacement sensor 210, a base 212, a supporting surface 214, control electronics 218, a computer 220 and a stop switch 222. FIG. 2A also illustrates a rat 226 as a subject and mesh supporting screen 224.

The subject 226 can be placed in a containment enclosure, such as a box, having a mesh screen 224 as a floor surface. Mesh screen 224 contains openings large enough to allow the tip of test filament 202 to pass there through while preventing the foot, or paw, of test animal 226 from passing through the screen 224. In a preferred embodiment, the enclosure is positioned so test filament 202 can push upward from beneath screen 224 to contact a test area on the surface of a paw of test animal 226.

System 200 uses a test filament 202 having a proximal end and a distal end. The distal end of test filament 202 is used to exert a force on a test area of the surface of a foot pad of test animal 226, while the proximal end is inserted into retainer 204. Retainer 204 operates to securely retain the proximal end of test filament 202. In addition, retainer 204 acts as an adapter allowing a transition from test filament 202 to motor shaft 205. Retainer 204 may include a tapered inner volume having a proximal end and a distal end with the proximal end being larger in diameter than the distal end. The proximal end of test filament 202 is inserted into the proximal end of the tapered inner volume and pushed toward the distal, or narrow, end of retainer 204. The tapered volume is designed so that test filament 202 is retained at a desired pressure somewhere between the large end and narrow end. Alternatively, retainer 204 may use a plurality of internal fingers that exert substantially equal pressure on the outer surface of the proximate end of test filament 202 when a collar is rotated, or otherwise closed.

Motor 206 is a linear motor that extends or retracts shaft 205 in response to signals received by way of data cable 216 from motor controller 218. In a preferred embodiment, the motor 206 is model H2W NCC02-05-005-4JBAT (H2W Technologies, Inc., Valencia, Calif.). Suitable motor controllers can be obtained from Galil Motion Control (Rocklin, Calif.). System 200 may also include a displacement measuring device for determining the amount, or length, of shaft 205 extending beyond motor 206. In the embodiment of FIG. 2A, the displacement sensor 210 is a linear variable differential transformer (LVDT, Trans-Tek model 0241-0000). Alternatively, the displacement sensor can be a linear encoder or a linear potentiometer. The longitudinal axes of motor 206, shaft 205 and test filament 202 are aligned parallel to axis 201.

A moveable base 212 is coupled to the lower end of displacement transducer 210 for facilitating movement of test filament 202 from one test location to another. While the embodiment of FIG. 2A employs a manually positioned base 212, alternative embodiments may employ bases that are moved by way of automated positioning devices such as, for example, robotic arms, actuators for positioning test filament 202 using a grid oriented coordinate system, etc. Surface 214 may be any surface capable of supporting manually positioned base 212 such as a bench top or desk top. A mirror 208 may be oriented substantially perpendicular to shaft 205 to facilitate positioning of filament 202 beneath animal 226. Alternatively, if the operator is located to the side of the containment enclosure, mirror 208 is oriented at an oblique angle to positioning of filament 202 with respect to the selected test location. Mirror 208 may also be used to facilitate observation of when animal 226 lifts a paw in response to filament 202.

Motor controller 218 operates under the control of a computer 220 and provides signals to motor 208 for causing shaft 205 to extend outward toward a test subject and to retract into the housing of motor 206, away from a test subject. An operator initializes system 200 when the distal end of test filament 202 is at a desired position with respect to a test animal 226. System 200, using computer 220, instructs motor controller 218 to cause shaft 202 to move towards animal 226. While shaft 202 moves, displacement sensor 210 measures the distance shaft 202 has traversed.

In the embodiment of FIG. 2A, encoder units are used to monitor the displacement of shaft 202. Motors having force outputs proportional to motor input currents are used so that force exerted by the motor can be determined from the supplied current. The force exerted by motor 206 is linearly increased by changing a torque limit setting. This causes motor 206 to exert a maximum allowable force through out the travel range of shaft 202. Shaft 202 may be extended to its maximum allowed limit by way of the current torque limit setting. Shaft 202 is displaced toward test animal 226 using this technique until the test animal 226 signals that it has sensed pain by lifting its paw in response to pressure applied by the distal end of test filament 202. System 200 is preferably also configured so as to facilitate rapid re-initialization for repetition of an experiment.

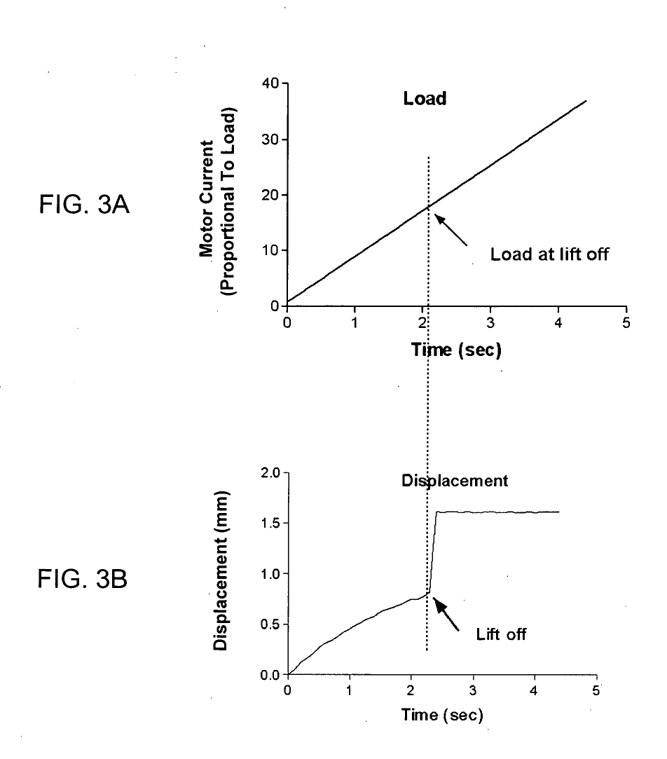

In use, manually positioned base 212 is placed on surface 214 and moved until position under a foot of the subject 226 with the foot in the path of filament 202. Closing foot switch 222 initiates a single trial in which the motor current is increased under control of the system 200 according to a pre-determined function, such as a linearly increasing ramp. Eventually the subject 226 lifts the foot, resulting in a sharp upward displacement of the filament 202. The data from such a trial is presented graphically in FIG. 3A and FIG. 3B. The sensory threshold is determined as the force (which is proportional to motor current) that was applied at the time that the foot was lifted, indicated by the dashed line in FIG. 3A and FIG. 3B.

FIG. 2B is a diagrammatic illustration of the relationship of linear motor 206, test filament retainer 204, displacement sensor 210, movable base 212 and cable 216 in one embodiment of the sensory test system of the present invention.

FIG. 3A and FIG. 3B illustrate, respectively, plots of load and displacement versus time for the embodiment used to gather data using a subject 226, here a laboratory animal such as a rat as shown in FIG. 2A. FIG. 3A shows the exerted load in motor current (which is proportional to load in grams) versus time. As seen from FIG. 3A, subject 226 lifted its paw in response to the distal end of test filament 202 shortly after two seconds. FIG. 3B shows that displacement of shaft 202 rapidly increased at about two seconds. The data shown in the lower plot corresponds to the data shown in the upper plot. The point at which a subject responds to stimuli applied by system 200 is referred to as a lift threshold 302.

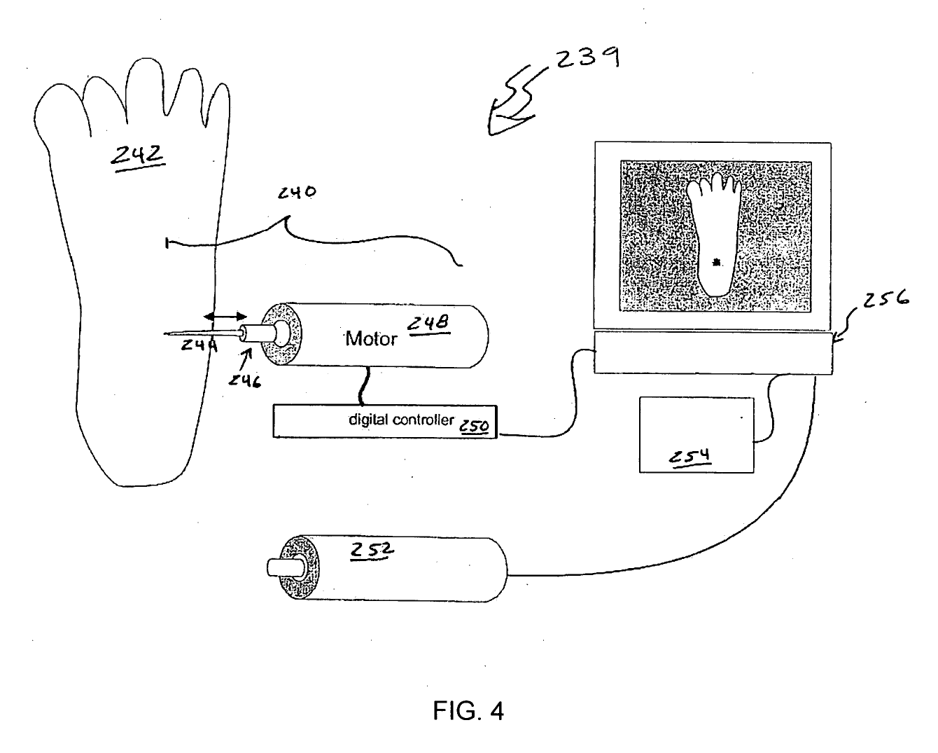

FIG. 4 illustrates a second preferred embodiment that employs a handheld probe 240 for performing sensory testing on human subjects. One embodiment of a system 239 is shown in FIG. 4, being used to make sensory measurements on, for example, a foot 242. System 239 includes a test filament 244, retainer 246, motor 248, motor controller 250, push button 252, foot switch 254, and computer 256. In other embodiments, the probe includes test filament 244, retainer 246, motor 248, motor controller 250, and computer 256 configured as a single handheld device.

An operator positions the handheld probe 240 at a desired location relative to foot 242. After initializing system 239, the operator activates foot switch 254 to begin advancement of test filament 244 toward foot 242. In preferred embodiments, test filament 244 is made of a naturally occurring or synthetic polymer. In other embodiments, test filament 240 can made of stainless steel or composite. Material, length and diameter of test filament 240 are selected to transmit force to produce accurate measurements. Test filament 240 is attached to the shaft of motor 248 by way of retainer 246. Motor controller 250 causes test filament 244 to advance in response to closure of foot switch 254 by increasing the motor current, and thus the force exerted by the filament, according to a pre-determined function, such as a linearly increasing ramp. Test filament 244 advances until the subject depresses push button 252. When push button 252 is closed by the subject, computer 256 ceases advancement of test filament 244 and records the applied force, ending the single trial.

Test filament 244 can be positioned at another location with respect to foot 242 and the measurement sequence repeated. With human subjects, or test subjects, instructions can be given with respect to when the push button 252 should be pressed relative to a perceived sensation. For example, a subject can be instructed to press the button as soon as any tactile sensation is perceived, or the subject can be instructed to push the button only when a certain level of pain is perceived.

Prior to performing testing, an image of test locations can be generated and displayed using computer 256. The image may be a standard template or may be generated using an overlay of a video image and the numerical results. When force measurements are obtained at test locations, the results can be numerically or graphically displayed at the corresponding location on computer 256. Using computer 256 in conjunction with handheld probe 240 thus lets an operator generate a real-time map of a subject's extremity using measured data. The mapped results can then be used to coordinate additional testing or to aid in diagnosis. In addition, the mapped results can be shown to the subject to facilitate his/her understanding of diagnosed conditions.

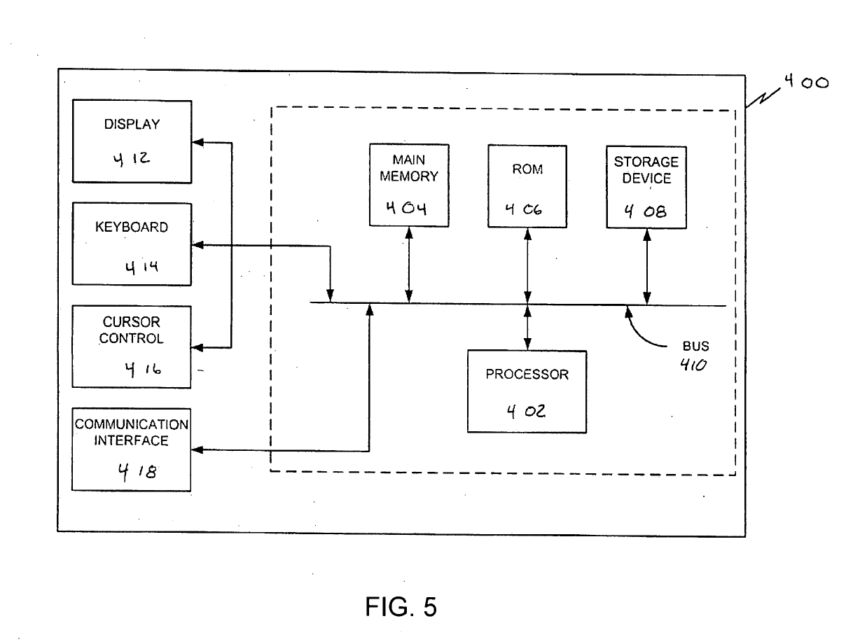

FIG. 5 illustrates an embodiment of computer 220, 256 in more detail as an exemplary computer 400. The exemplary computer 400 includes a processor 402, main memory 404, read only memory (ROM) 406, storage device 408, bus 410, display 412, keyboard 414, cursor control 416, and communication interface 418.

Processor 402 may be any type of conventional processing device that interprets and executes instructions. Main memory 404 may be a random access memory (RAM) or a similar dynamic storage device. Main memory 404 stores information and instructions to be executed by processor 402. Main memory 404 may also be used for storing temporary variables or other intermediate information during execution of instructions by processor 402. Main memory 404 may also be used for storing temporary variables or other intermediate information during execution of instructions by processor 402. ROM 406 may be replaced with some other type of static storage device. Data storage device 408 may include any type of magnetic or optical media and its. corresponding interfaces and operational hardware. Data storage device 408 stores information and instructions for use by processor 402. Bus 410 includes a set of hardware lines (conductors, optical fibers, or the like) that allow for data transfer among the components of computer 400.

Display device 412 may be a cathode ray tube (CRT), liquid crystal display (LCD) or the like, for displaying information to a user. Keyboard 414 and cursor control 416 allow the user to interact with computer 400. Cursor control 416 may be, for example, a mouse. In an alternative configuration, keyboard 414 and cursor control 416 can be replaced with a microphone and voice recognition software to enable the user to interact with computer 400.

Communication interface 418 enables computer 400 to communicate with other devices/systems via any communications medium. For example, communication interface 418 may be a modem, an Ethernet interface to a LAN, or a printer interface. Alternatively, communication interface 418 can be any other interface that enables communication between computer 400 and other devices or systems.

By way of example, a computer 400 suitable for use in an embodiment of the present invention provides control to a motor driven cutaneous indentation sensory testing device described elsewhere in this disclosure. Computer 400 performs operations necessary to complete desired actions in response to processor 402 executing sequences of instructions contained in, for example, memory 404. Such instructions may be read into memory 404 from another computer-readable medium, such as a data storage device 408, or from another device via communication interface 418. Execution of the sequences of instructions contained in memory 404 causes processor 402 to perform a method for extending a testing sensor until a determined pressure is exerted on a subject's skin and for recording the exerted pressure when a subject provides notification to an operator. For example, processor 402 may execute instructions to perform the functions of measuring cutaneous sensory activity. Alternatively, hard-wired circuitry may be used in place of or in combination with software instructions to implement the present invention. Thus, the present invention is not limited to any specific combination of hardware circuitry and software.

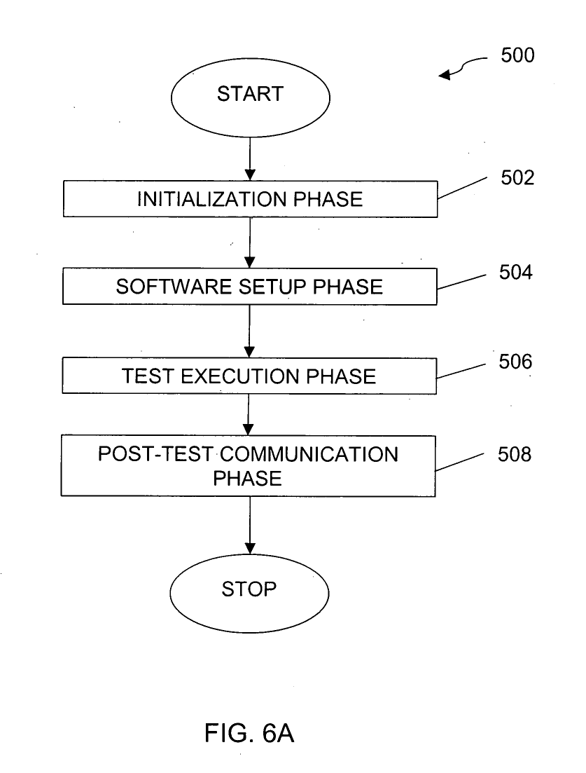

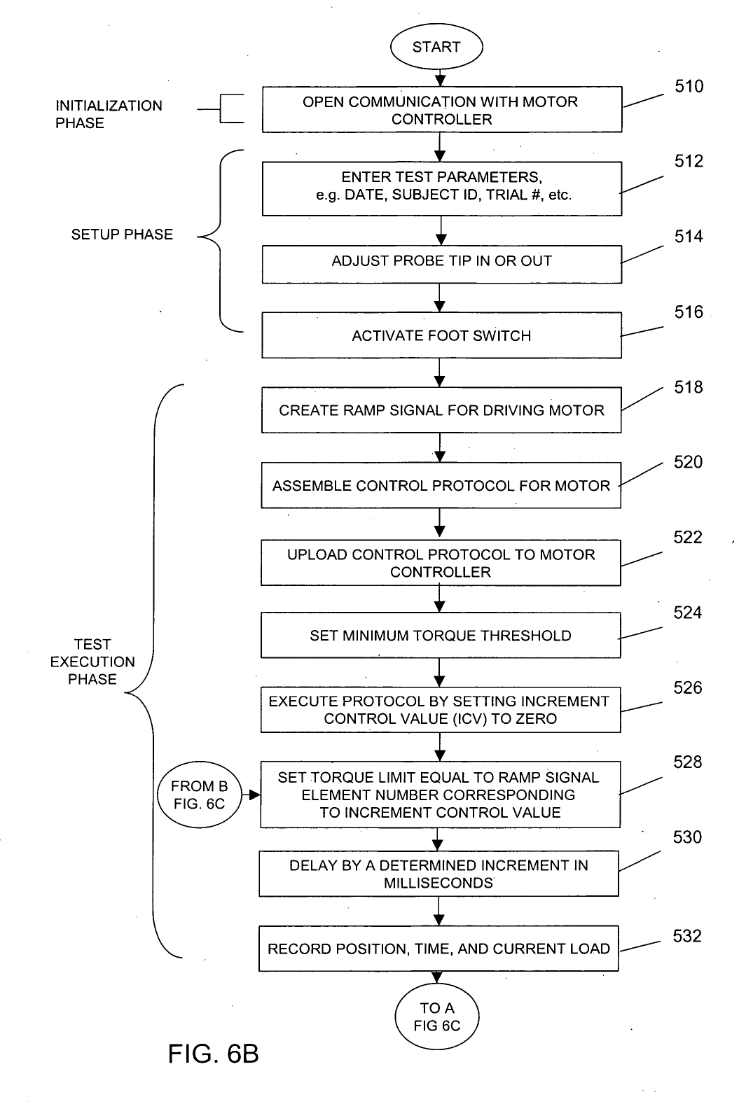

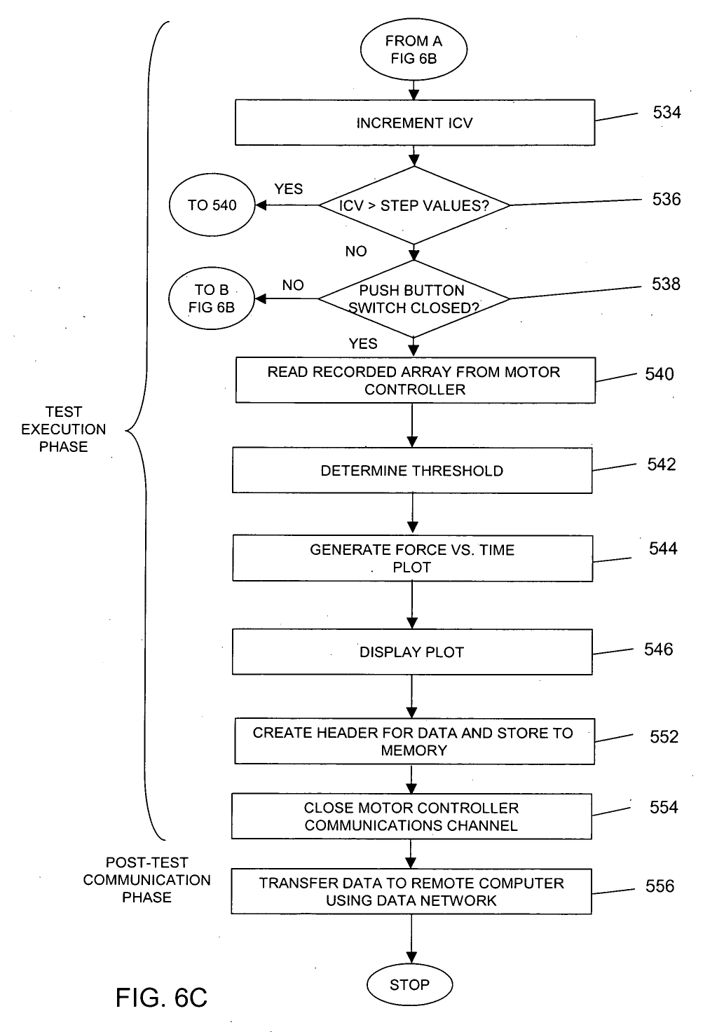

FIG. 6A provides a high level flow diagram showing four phases associated with an exemplary method for using system 239 to perform sensory testing on a subject. The method begins with an initialization phase which involves supplying power to components such as motor controller 250 (per step 502). Next software is set up (per step 504) and then one or more sensory tests are performed (per step 506). When testing is complete, results may be transferred to other devices and systems (per step 508).

FIGS. 6B and 6C together illustrate the steps of FIG. 6A in detail. Communication with motor controller 250 is opened (per step 510). Test parameters are then entered into computer 256 using keyboard 414 (per step 512). Examples of test parameters are, but are not limited to, date and time of test, name of ID of test subject, area of body being tested, upper limit of force to be used, insurance provider information, operator's name and ramping rate for the motor drive signal. Then probe 244 is adjusted in or out with respect to motor 248 (per step 514).

Footswitch 254 is then operated to start the experiment (per step 516). In response to the signal from footswitch 254, the ramp signal for driving motor 248 is generated (per step 518) with a number of step values, determined in step 512. Next, the appropriate control protocol for motor 248 is assembled (per step 520) and uploaded to motor controller 250 (per step 522). A minimum torque threshold for shaft 205 is set (per step 524). Next, the control protocol is executed by setting the iteration count value (ICV) to zero (per step 526). Then the torque limit is set to an element equal to the ramp signal value (SV), whose step value corresponds to the current iteration count value (per step 528). Movement of probe 244 is then delayed by a determined increment (per step 530). For example, advancement of probe 244 may be delayed by 100 milliseconds (ms), 500 ms, or 1000 ms. The position of probe 244 is measured along with the applied current and the present time (per step 532).

Now referring to FIG. 6C, the iteration count value is advanced incrementally, (per step 534). If a subject senses the distal end of probe 244 in response to its advancement (per step 536), the subject depresses push button switch 252 (per step 538). In contrast, if the subject does not sense probe 244, the method returns back to step 528 (FIG. 6B) from step 536. A safety measure is built in for unresponsive subjects wherein the system stops if the iteration count value exceeds the number of step values in the ramp signal.

When the subject closes push button switch 252, or if the ramp is ended, a signal is received at computer 256. Receipt of the signal causes computer 256 to generate a force vs. time plot. The force vs. time plot is then displayed on display 412 (per step 544), and the threshold is taken as the force at the time the push button switch 252 was closed.

Computer 256 then creates a header for the data generated during the experiment and stores the acquired data and header as a file in memory (per step 552). The header contains information about the gathered data such as the date, subject's name, system settings and the like. The above sequence can repeated at at one or more additional locations. Then the motor communications channel is closed (per step 554). After storing the acquired data and ceasing communication with motor controller 250, computer 256 may transfer the file to another device or system using a data network (per step 556). If a map is being plotted, another position is selected and the above sequence is repeated. When the desired number of sites have been tested, the threshold data are displayed superimposed on the graphic representation of the test area, such as the sole of a foot.

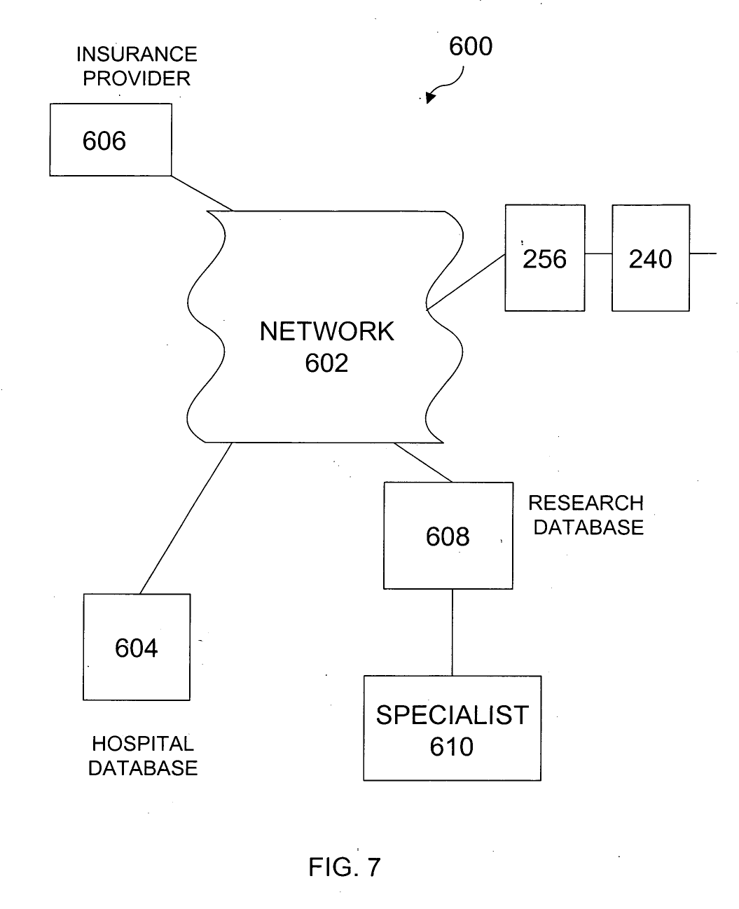

FIG. 7 illustrates a networked embodiment of the sensory testing system. Networked system 600 may include computer 256 and probe 240, an insurance provider server 606, a specialists' workstation 610 and a data network 602. After completing an experiment, an operator can instruct computer 256 to transmit acquired data to an insurance provider's server 606 using network 602. The insurance provider may use the data for authorizing additional treatment, for compiling statistics on its insured population, and for performing its own analysis. Computer 256 may also transmit acquired data to a hospital database 604 for permanent storage and to provide access to other departments within the hospital. Computer 256 may also transmit data to a research database 608.

Research database 608 may be used to support one or more ongoing studies involving the sensory perception of animals and/or human subjects. Research database 608 may be coupled to a specialist's computer 610. Specialist's computer 610 may be operated by a person having a high level of expertise in a field that is pertinent to the acquired data. For example, the specialist may be responsible for running and overseeing experiments or he/she may be skilled at making diagnoses based on the data.

Network 602 may be any type of communications network employing any type of networking protocol. For example, network 602 may be an internet protocol (IP) network, an asynchronous transfer mode (ATM) network, or conventional telephone network such as a plain old telephone system (POTS) network.

The claims should not be read as limited to the described order or elements unless stated to that effect. Therefore, all embodiments that come within the scope and spirit of the following claims and equivalents thereto are claimed as the invention.

What is claimed:

1. A system for performing sensory testing on a living subject, the system comprising:
a test filament having a proximal end and a distal end, the proximal end for engaging a test subject, the test filament further associated with an axis substantially parallel to the probe;
a motor having a shaft moveable along the axis, the shaft further coupled to the distal end of the test filament, the motor further capable of moving the shaft at a constant force without requiring a force measuring device in communication with the test filament; and
a controller for directing the operation of the motor.

2. The system of claim 1 wherein the test filament remains substantially rigid over a determined range of applied forces.

3. The system of claim 1 wherein the motor is a linear motor.

4. The system of claim 3 further comprising:
a displacement sensor for measuring a displacement of the shaft.

5. The system of claim 3 wherein the controller is a computer processing machine-readable instructions.

6. The system of claim 3 wherein the subject is a human.

7. The system of claim 4 wherein the subject is nonhuman.

8. The system of claim 5 further comprising a foot switch for initiating the sensory test.

9. The system of claim 8 further comprising a second switch for stopping movement of the shaft.

10. The system of claim 9 wherein the computer displays a first plot showing a load versus time and further displays a second plot showing a displacement versus time.

11. A method for performing sensory measurements on a living subject, the method comprising the steps of:
receiving an initialization instruction from a controller;
receiving an advancement instruction for causing a shaft to increment the force until a detection signal is received;
receiving the detection signal;
stopping advancement of the shaft in response to the detection signal; and
recording the operating force.

12. The method of claim 11 wherein the force corresponds to a force applied to the subject.

13. The method of claim 12 wherein the advancement instructions are received from a controller.

14. The method of claim 13 wherein the detection signal is a switch closure.

15. The method of claim 14 wherein the switch is controlled by an operator.

16. The method of claim 15 wherein the switch is controlled by the subject.
